(19) United States
(12) Patent Application Publication
(10) Pub. No.: US 2025/0072876 A1
(43) Pub. Date: Mar. 6, 2025

(54) MINIATURIZED BIMODAL OXYGEN MONITORING WEARABLE DEVICE

(71) Applicant: Worcester Polytechnic Institute, Worcester, MA (US)

(72) Inventors: Ulkuhan Guler, Shrewsbury, MA (US); Vladimir Vakhter, Worcester, MA (US); Burak Kahraman, Worcester, MA (US)

(21) Appl. No.: 18/950,028

(22) Filed: Nov. 16, 2024

Related U.S. Application Data
(63) Continuation-in-part of application No. 18/924,362, filed on Oct. 23, 2024.
(63) Continuation-in-part of application No. 17/960,105, filed on Oct. 4, 2022.
Application No. 17/960,105 is a continuation of application No. 17/066,570, filed on Oct. 9, 2020, now Pat. No. 11,849,923.
(60) Provisional application No. 63/545,273, filed on Oct. 23, 2023.
(60) Provisional application No. 63/599,825, filed on Nov. 16, 2023.
(60) Provisional application No. 63/252,251, filed on Oct. 5, 2021.
(60) Provisional application No. 62/913,299, filed on Oct. 10, 2019.

(51) Int. Cl. A61B 10/00; A61B 5/00; A61B 5/145

(52) U.S. Cl. CPC A61B 10/00; A61B 5/14542; A61B 5/443; A61B 5/6832; A61B 2010/0083

(57) ABSTRACT

A miniaturized wireless bimodal oxygenation status monitoring wearable device that continuously monitors both the transcutaneous oxygen and peripheral blood oxygen saturation and overcomes the limitations of the traditional transcutaneous oxygen monitors such as requiring a heating element and a large, expensive bedside monitor that prevents continuous monitoring outside a clinical setting.

### RELATED APPLICATIONS

This patent application claims the benefit under 35 U.S.C. § 119 (e) of U.S. Provisional Patent App. No. 63/599,825, filed Nov. 16, 2023, entitled “WEARABLE OXYGEN MEASUREMENT DEVICE,” and is a Continuation-in-Part (CIP) under 35 U.S.C. § 120 of U.S. application Ser. No. 17/960,105, filed Oct. 4, 2022, entitled “TRANSCUTANEOUS GASEOUS MEASUREMENT DEVICE,” which is a Continuation-in-Part (CIP) under 35 U.S.C. § 120 of U.S. patent application Ser. No. 17/066,570, filed Oct. 9, 2020, entitled “WEARABLE BLOOD GAS MONITOR,” which claims the benefit under 35 U.S.C. § 119 (e) of U.S. Provisional Patent App. No. 62/913,299, filed Oct. 10, 2019, entitled “WEARABLE BLOOD GAS MONITOR,” and is also a Continuation-in-Part (CIP) under 35 U.S.C. § 120 of U.S. patent application Ser. No. 18/924,362 filed Oct. 23, 2024, entitled “CHARGE-TO-DIGITAL CONVERTER FOR TIME-DOMAIN DUAL LIFETIME REFERENCING,” which claims the benefit under 35 U.S.C. § 119 (e) of U.S. Provisional Patent App. No. 63/545,273, filed Oct. 23, 2023, entitled “TRANSCUTANEOUS GASEOUS MEASUREMENT DEVICE,” all incorporated herein by reference in entirety.

### BACKGROUND

Blood gas testing is an effective tool for analysis and diagnosis of many human physiological parameters. Blood chemistry can be an effective indicator of many bodily functions and conditions. Among the vital signs of human body, respiration is a key component of a person's health. Respiratory health can be quantified by rate, volume, and blood-gas content. Traditional respiration monitoring methods such as arterial blood gas monitoring and pulse oximetry have certain advantages and disadvantages.

### SUMMARY

A transdermal patch measures a gaseous concentration based on transcutaneous diffusion through an epidermal surface of a patient. Transcutaneous oxygen and carbon dioxide differ from a measurement of saturated gases often measured in a patient blood flow or tissue. The patch employs an indicator responsive to an oxygen presence for emitting light having an intensity and lifetime based on the gaseous presence. An optical receptor is in communication with logic for receiving the intensity and lifetime (i.e. duration) of emitted light and computing the oxygen saturation or pressure level in a bimodal manner for SpO₂ and PtcO₂. Low power demands and circuit footprint are amenable to a wearable device such as a patch for continuous use.

The device may take the form of a bimodal oxygenation status monitoring wearable device that will continuously monitor both the transcutaneous oxygen and peripheral blood oxygen saturation and will overcome the limitations of the traditional transcutaneous oxygen monitors such as requiring a heating element and a large, expensive bedside monitor that prevents continuous monitoring outside a clinical setting.

Configurations herein are based, in part, on the observation that oxygen sensing is frequently employed in many physiologic contexts due to its prevalent nature in essential human respiration. Oxygen in various forms and concentrations is found abundantly throughout living tissue, and therefore is often a reliable indicator of proper and healthy physiology. Unfortunately, conventional approaches to oxygen monitoring rely on oxygen saturation in blood, relating to hemoglobin-bound oxygen (oxyhemoglobin) in oxygenated blood. Measurement of oxygen concentration in tissue conventionally requires a robust apparatus with heating and power requirements inconsistent with a wearable device. Oxygen concentration measures the oxygen concentration based on partial pressure of oxygen dissolved in the arterial bloodstream, and is often preferred to a measurement of oxygen saturation, or may be taken in conjunction with saturated O₂.

Accordingly, configurations herein substantially overcome the shortcomings of conventional, bulky oxygen detection by providing a wearable oxygen sensor in the form of a patch or epidermal appliance for measuring transcutaneous oxygen upon diffusion through the epidermal surface. A photoluminescent indicator emits light responsive to an illuminating stimuli in a manner that varies with the presence of the diffused oxygen. An electronic circuit performs computations and implements logic for determining the oxygen concentration of the underlying tissue based on the partial pressure of the oxygen computed by the system.

In further detail, configurations below disclose an epidermal, portable, wireless transcutaneous sensing device for bimodal sensing of SpO₂ and PtcO₂, including a saturation sensor configured to detect a peripheral blood oxygen saturation (SpO₂) based on an optical response from the skin surface, and a transcutaneous sensor configured to detect a partial pressure of transcutaneous oxygen (PtcO₂) based on an optical response from a skin surface. Portability is provided by a wireless transmitter for communication with a remote server configured for rendering values corresponding to the transcutaneous oxygen and peripheral blood oxygen saturation, without a need for a heating element or large bedside monitor.

In a particular configuration directed to oxygen on the epidermal surface, the optical source emits light in a blue spectrum, and the photodetector is sensitive to light in a red spectrum. The sensing film may be a luminescent sensing film adapted for adherence to an epidermal surface. In one configuration, the luminescence of the luminophore functional groups is quenched in the presence of oxygen, reducing the intensity and lifetime of the re-emitted red light. Change in the intensity and lifetime of the re-emitted red light received by the photodetector is inversely proportional to the change in the concentration of oxygen based on the partial pressure of transcutaneous oxygen (PtcO₂) diffusing from the epidermal surface. The wavelength sensitivity is specific to particular luminophores in the luminescent sensing film, as is the remitted light. Alternative sensing films may involve different wavelengths.

In contrast to conventional approaches, the PtcO₂ sensor and associated logic is encapsulated in a wearable patch or low-profile, adhesive arrangement and includes a wireless transmitter for offloading storage and analytics capability. While conventional SpO₂ measurement has been achieved in finger-attached wired sensors, compact sensing of PtcO₂ levels has typically required a larger footprint device including a heating element for electrochemical sensing of perfusion. Therefore, conventional approaches employ an electrochemical, rather than optical measurement medium and are not feasible for miniaturization due to the power demands of the heating element, calibration and computation units.

Those skilled in the art should readily appreciate that the programs and methods defined herein are deliverable to a user processing and rendering device in many forms, including but not limited to a) information permanently stored on non-writeable storage media such as ROM devices, b) information alterably stored on writeable non-transitory storage media such as solid state drives (SSDs) and media, flash drives, floppy disks, magnetic tapes, CDs, RAM devices, and other magnetic and optical media, or c) information conveyed to a computer through communication media, as in an electronic network such as the Internet or telephone modem lines. The operations and methods may be implemented in a software executable object or as a set of encoded instructions for execution by a processor responsive to the instructions, including virtual machines and hypervisor controlled execution environments. Alternatively, the operations and methods disclosed herein may be embodied in whole or in part using hardware components, such as Application Specific Integrated Circuits (ASICs), Field Programmable Gate Arrays (FPGAs), state machines, controllers or other hardware components or devices, or a combination of hardware, software, and firmware components.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIGS. 1A and 1B are context diagrams including a wearable sensor patch suitable for use with configurations herein;

FIG. 2 shows a side cutaway view of the wearable patch of FIG. 1 in communication with an epidermal surface of a patient;

FIG. 3 shows a schematic view of a light intensity and processing circuit for the wearable patch of FIG. 2;

FIG. 4 is a diagram of light emission energy received by the circuit of FIG. 3;

FIG. 5 is a graph of the light wavelengths emitted in the diagram of FIG. 4;

FIG. 6 is a block diagram of the wearable patch as described in FIGS. 3-5;

FIGS. 7A-7D show graphs of voltage and light pulses in the circuit of FIG. 6;

FIGS. 8A-8C shows a further detail of the analog front end of FIG. 6;

FIG. 9 shows a relation between intensity and lifetime (duration) of remitted light;

FIGS. 10 and 11 demonstrate the luminescence lifetime measured with the prototype monitor for different partial pressures of oxygen;

FIG. 12 shows the transient response of the PtcO₂ monitor to the step changes in the partial pressure of oxygen;

FIGS. 13 and 14 show a measured lifetime at the fingertip during an occlusion event for several test subjects, respectively; and

FIG. 15 shows a measured lifetime on the fingertip during a hypoxia event.

### DETAILED DESCRIPTION

The description below presents an example of a wearable device for measurement of oxygen (O₂) by transcutaneous partial pressure (PtcO₂) and saturated oxygen (SpO₂), which differs from conventional wearable measurement because conventional approaches solely measure saturated oxygen (SpO₂). Saturated oxygen is bound to hemoglobin, while PtcO₂ measurement refers to a concentration of total oxygen. Depending on the medical context, PtcO₂ based readings have an advantage over conventional SpO₂, either alone or in conjunction with SpO₂ readings.

A blood gas measurement device includes an optical source and a sensing film adapted for adherence to an epidermal surface and responsive to gaseous diffusion from the epidermal surface. The sensing film is sensitive to the gas or gases targeted for sensing based on re-emittance properties that are affected by transdermal gaseous diffusion. An optical source, typically an LED for low power and heat properties, emits a light directed to the sensing film. A photodetector is sensitive to re-emitted light from the sensing film based on the optical source, and logic responsive to a signal from the photodetector computes a level of a blood gas based on the re-emitted light.

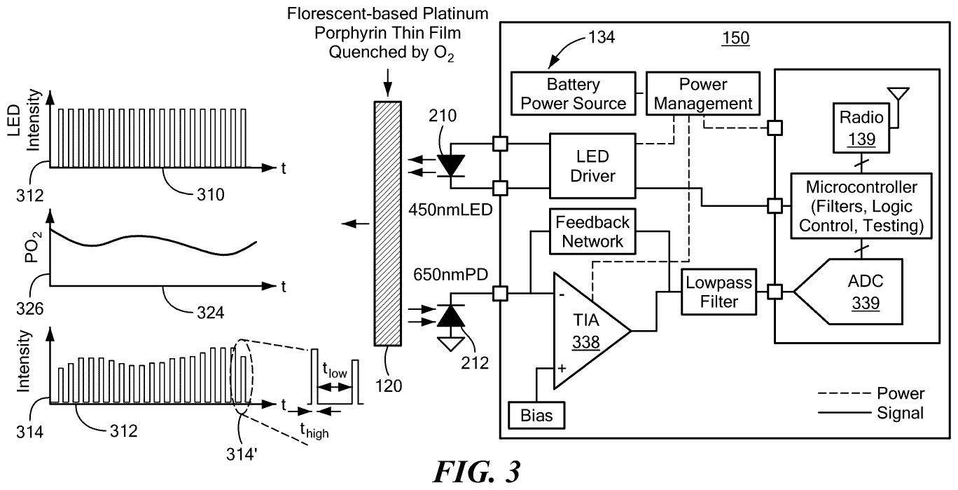

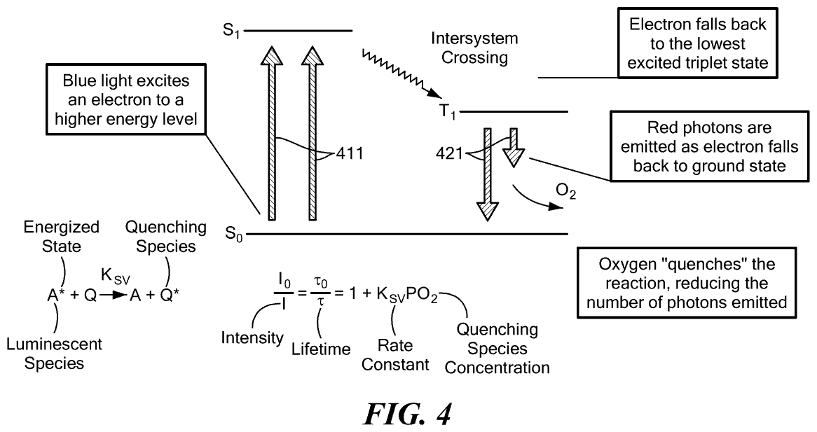

Photoluminescence refers to the emission of photons produced in certain molecules during de-excitation and is one of the possible physical effects resulting from the interaction between light and matter. When a luminescent molecule absorbs a photon, it is excited from a ground state to some higher energy level, and emits light upon its return to the lower state. The subsequent de-excitation processes are depicted below in FIGS. 4 and 5.

In the presence of molecular oxygen, the photoluminescence of such molecules is quenched via a radiationless deactivation process which involves molecular interaction between the quencher and the luminophore (collisional quenching) and it is therefore diffusion limited. The mechanism by which oxygen quenches luminescence is not germane to the disclosed approach, however it has been suggested that the paramagnetic oxygen causes the luminophore to undergo intersystem crossing to the triplet state while molecular oxygen goes to the excited state) and then returns to ground state.

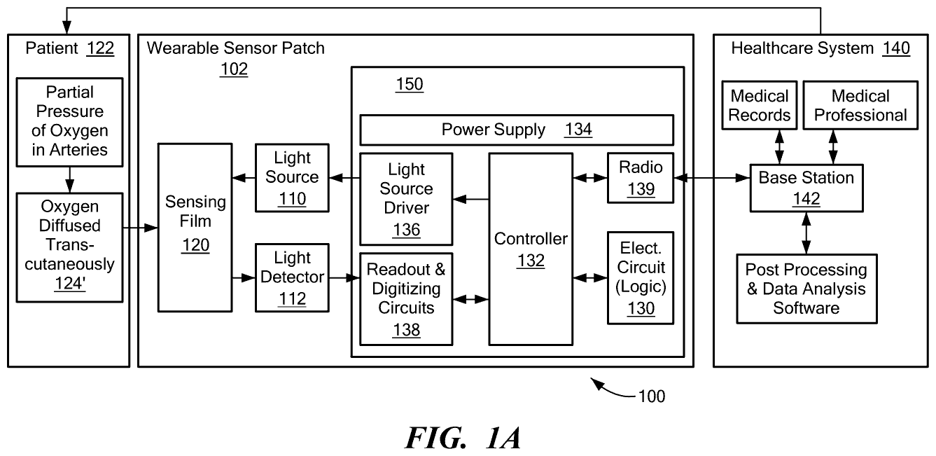

FIGS. 1A and 1B are context and system diagrams of wearable systems for transcutaneous oxygen and bimodal sensing of transcutaneous and saturated oxygen for wireless patient monitoring.

FIG. 1A is a system context diagram including a wearable sensor patch suitable for use with configurations herein. Referring to FIG. 1, a blood gas measurement device 100 includes an optical source 110 and a light sensitive medium 120 such as a sensing film. The light sensitive medium 120 is configured to emit light responsive to the optical source 110, in which the emitted light is based on a gaseous diffusion through the light sensitive medium 120. A photoreceptor 112 is disposed for receiving the emitted light, and logic 130 coupled to the receptor 112 computes a quantity of the gaseous diffusion based on the emitted light.

FIG. 1B shows a context diagram of an alternate configuration for combined partial pressure of oxygen (PtcO₂) and peripheral blood oxygen saturation (SpO₂);

Referring to FIG. 1B, an epidermal, portable wireless transcutaneous sensing device 200 includes a CMOS (complementary metal-oxide semiconductor) or similar semiconductor structure in a system on chip (SoC) form factor supporting a saturation sensor 250 configured to detect a peripheral blood oxygen saturation (SpO₂) based on an optical response from the skin surface 126-1. The device 200 also includes a transcutaneous sensor 230 supported by the SoC configured to detect a partial pressure of transcutaneous oxygen (PtcO₂) based on an optical response from a skin surface 126-2 (126 generally). A transmitter 232 is configured for communication with a remote server 234 configured for rendering values corresponding to the transcutaneous oxygen (PtcO₂) and peripheral blood oxygen saturation (SpO₂) via a wireless link 237. Network intermediaries such as a base station 236 or local router/wireless access point, and a cloud service 238 for Internet or similar network connectivity may be employed.

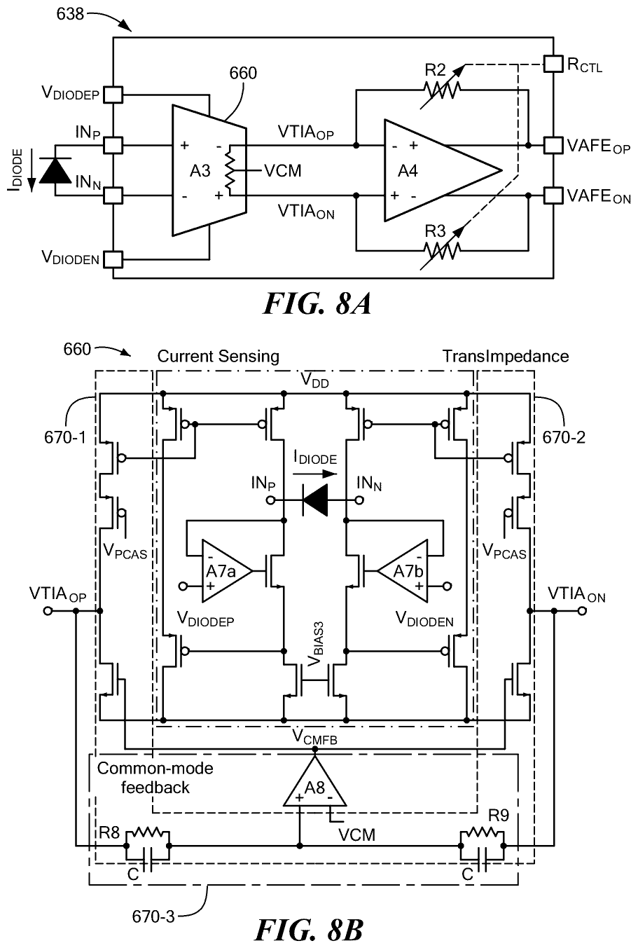

In the example configuration of FIG. 10, the saturation sensor 250 further comprises a plurality of illumination sources 254-1 . . . 254-2 (254 generally), and a plurality of photodetectors 252-1 . . . 252-2 (252 generally). A system-on-chip implementation 240 includes sensing logic 242 configured to activate the illumination sources 254 to irradiate the skin surface 126-1, monitor the photodetectors to receive reflected illumination, and compute the peripheral blood oxygen saturation 255 based on the reflected illumination.

The skin mounted form factor of the SoC implementation 240 further includes a substrate 241 configured for epidermal mounting, a first circuit 251 including the saturation sensor 250, and a second circuit 231 including the transcutaneous sensor 230. A power supply 244 such as a Li-ion battery connects to the first and second circuits 251, 231 and the transmitter 232. In the example form factor, the system on chip (SoC) implementation 240 contains the first circuit 251, the second circuit 231 the transmitter 232, and the logic 242.

The example configuration of the saturation sensor 250 includes a red LED (light emitting diode) and an infrared (IR) LED defining the illumination sources 254, and two photodetectors 252. The photodetectors 252 are configured for measuring respective resulting photoplethysmography (PPG) signals. Photoplethysmography (PPG) refers to an optically obtained plethysmogram that can be used to detect blood volume changes in the microvascular bed of tissue, and is computed directly or indirectly from the peripheral blood oxygen saturation 255 and transcutaneous partial pressure 235 sent to the server 234. In particular, the sensing logic 242 is configured to excite the red LED and the IR LED for inducing reflections based on metabolic variations in the skin surface, where the two photodetectors 252 are responsive to the reflections.

The device 200 employs the wireless link 237 for an interface to a public access network. The remote server 234 is accessible via the public access network and is further configured for storing and coalescing a series of respective values indicative of the SpO₂ and the PtcO₂ for generating an indication of respiratory health.

The device of FIG. 1B uses a luminescent film to sense PtcO₂. The system measures PtcO₂ using an optical method, as above in FIGS. 1-9. The luminescent sensing film 120 on the wearable patch is placed on the patient, typically an accessible epidermal surface such as a wrist. The film 120 is exposed to light from a light source such as a light-emitting diode (LED). The lifetime of re-emitted light from the film will be dependent on the concentration of the parameter (level of oxygen) being measured. The change in the lifetime of the re-emitted light is measured by a light detector such as a photodiode (PD). However, in further contrast to conventional approaches, the system also measures SpO₂ using an optical approach. The system includes two additional light sources-red and infrared (IR) LEDs-and two additional light detectors (other PDs) in a reflective configuration for the measurement of SpO₂. The system excites the two light sources independently and measures two different resulting photoplethysmography (PPG) signals, that carry the necessary information for the calculation of SpO₂, with the light detectors. The system-on-chip (SoC) on the device controls the functioning of the light sources and measures the corresponding changes detected by the light detectors. The measured values are transmitted using a wireless interface to a base station/controller serving as a gateway securely transmitting it to a cloud for post processing, data analysis, and storage and displaying the processed data.

Implementation is well suited to a wearable device in a suitable form factor where the sensor (LEDs, PDs, and sensing film) is integrated onto a substrate along with the required electrical components-a custom CMOS system-on-chip (SoC) or off-the-shelf integrated circuits (ICs)-for measurement of PtcO₂ and SpO₂, control, and processing. The CMOS SoC 240 integrates the readout circuitry, digital logic circuits, and wireless communications into one small, low power package that is comfortable for long-term wear. The system can do analysis of blood gas content of oxygen by measuring two vital parameters, namely PtcO₂ and SpO₂, giving a holistic view of the wellbeing of the patient. The system could be powered by either a wireless power link or a battery. The system would communicate using a radio with standard protocols such as Bluetooth Low Energy (BLE), Wi-Fi, ZigBee, or a customized low power protocol that could be coupled with the wireless power link. The system could also save data on the board in a non-volatile memory persistently or pending transmission via the wireless link 237.

In an example configuration, the device 100 takes the form of a wearable patch 102 adhered to or adjacent to the skin (epidermal surface) of a patient 122 for disposing a sensing circuit 150 thereon. An electronic circuit and/or processor instruction sequence implements the logic 130 which computes the oxygen concentration based on the duration and intensity of the emitted light. The photoreceptor 112 receives the light, and readout and digitizing circuits 138 transform and digitize the received light into intensity and lifetime (duration) values employed by the logic 130. A controller 132 is driven by a power supply 134 for powering the readout and digitizing electronics 138, controller 132, radio 139 and a light source driver 136 activating the LED light source 110. The controller 132 couples to the readout and digitizing circuit 138 for receiving emitted light. A radio 139 wirelessly couples to a base station 142 for gathering and transporting the computed oxygen levels, and may take the form of a personal device, hospital monitor, data logger or any suitable network device for capturing the computed oxygen levels and related data without encumbering the patient 122 with bulky devices. The patient data may then be recorded, stored and analyzed according to the patient's healthcare regimen. In contrast to conventional tethered approaches which require an electronic connection (wire), the radio 139 implements a wireless connection to a monitoring base station 142 for receiving and coalescing patient data and generating needed alerts and reports resulting from the oxygen concentration.

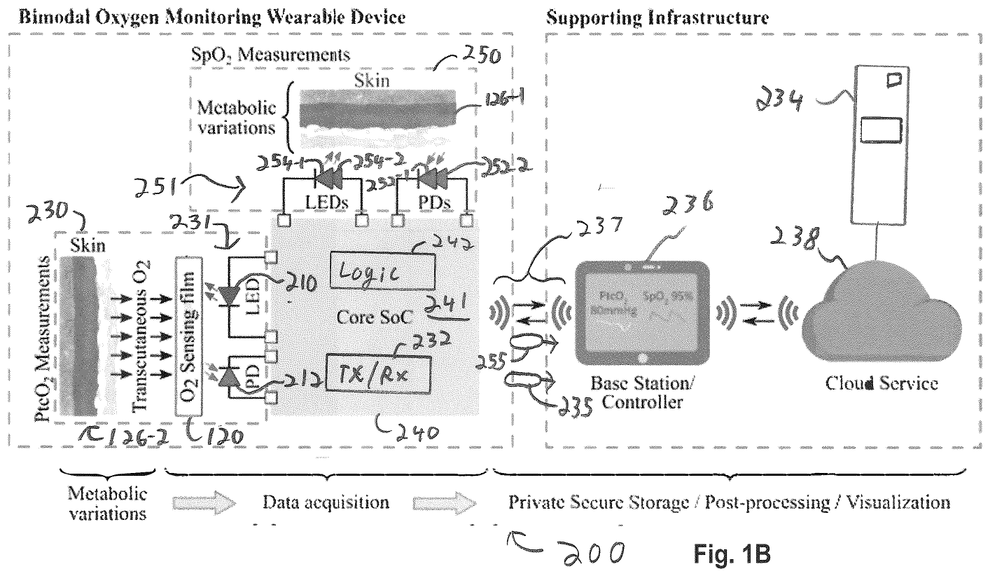

FIG. 2 shows a side cutaway view of the wearable patch 102 of FIG. 1 adhered to an epidermal portion 104 in communication with an epidermal surface 126 of the patient 122. Referring to FIGS. 1 and 2, the light sensitive medium 120 takes the form of a luminescent sensing film 120′ adapted for communication with a gaseous diffusion source for receiving the gaseous diffusion. Diffused gases 124 including oxygen diffuses through dermal layers 122′ of the patient 122 and through the sensing film 120. Subcutaneous tissue 105, derma 107 and epidermis 109 define the dermal layers 122′ that are the source of the transcutaneously diffused oxygen 124′. The sensing film 120 is maintained in communication with the epidermal surface 126 by a collection medium adapted to engage a surface for coupling the light sensitive medium to a source of the gaseous diffusion. The collection medium may take the form of an adhesive, taped, or strapped patch for directing the diffused gases 124 from the surface to the collection medium.

For example, in a particular use case, collection medium is a planar epidermal patch 102 adapted for receiving gaseous diffusion from an infant 101 patient. The ability to remotely monitor infants could improve the feasibility of early discharge and reduce the risk of undiagnosed issues becoming significant after hospital release. Continuous and accurate remote tracking of vital respiratory parameters in a fully wireless manner could provide relevant and accurate data to the caregiver to inform the course of treatment. Configurations herein address the need to monitor patience's transcutaneous oxygen level remotely and safely by a medical professional with a light and low-height profile wearable device. Conventional vital monitoring systems, especially those that monitor blood gas status are typically large, bulky bed-side machines with wired electrodes and are usually used in a hospital setting. These machines require the patient to be tethered to a hospital bed with limited mobility.

Dermal placement of the patch 102 can be made elsewhere such as the abdomen or torso where it is less susceptible to patient movement, further enhanced by the omission of wired tethers. Such a patch encloses the light sensitive medium 120 in a sealing engagement with the dermal surface 126 for quantifying the transdermal oxygen emitted or diffused through the patch.

It its most basic form, the patch 102 and sensing device 100 perform a method for sensing an oxygen concentration based on transcutaneous oxygen 124′ by receiving a diffusion of oxygen 124′ and other gases 124 through the transcutaneous surface 126. The patch 102 adheres the light sensitive medium 120 to the transcutaneous surface 126, such that the light sensitive medium 120 has a photoluminescent response to the diffused oxygen 124′. A pulsed light 111 from the light source 110 on the light sensitive medium 120 causes the photodetector 112 to receive an emitted light 121 in response to the diffused oxygen. The logic 130 computes the partial pressure of oxygen value based on an oxygen sensitive luminophore in the light sensitive medium 120 responsive to quenching of the emitted light 121 inversely with the oxygen presence. In other words, the emitted light “fades” faster with greater oxygen, now discussed in more detail below.

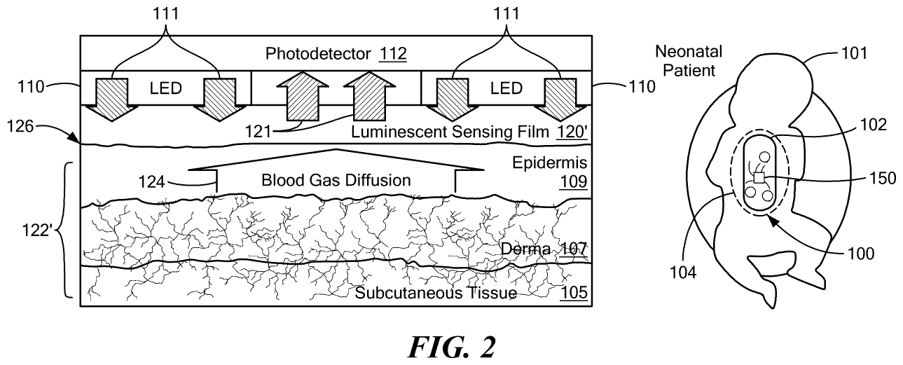

FIG. 3 shows a schematic view of a light intensity and processing circuit for the wearable patch of FIG. 2. Referring to FIGS. 1-3, as described above, the light sensitive medium 120 is a sensing film adapted for adherence to an epidermal surface 126 and responsive to gaseous diffusion from the epidermal surface. In FIG. 3, the device 100 is shown in a schematic of the fluorescent-based transcutaneous oxygen sensing system.

A low power transimpedance amplifier and low power microcontroller connect to wireless system and powered by a coin cell battery. Alternate configurations may employ any suitable power arrangement, such as a coin cell, thin film battery, wireless power link, or similar methods. The disclosed system communicates to a base station 142 using a wireless protocol, such as Bluetooth®, Bluetooth LE, ZigBee®, WiFi, NFC (Near Field Communication) or similar approach consistent with FDA (Food and Drug Administration) and other governmental recommendations or guidelines for health related devices.

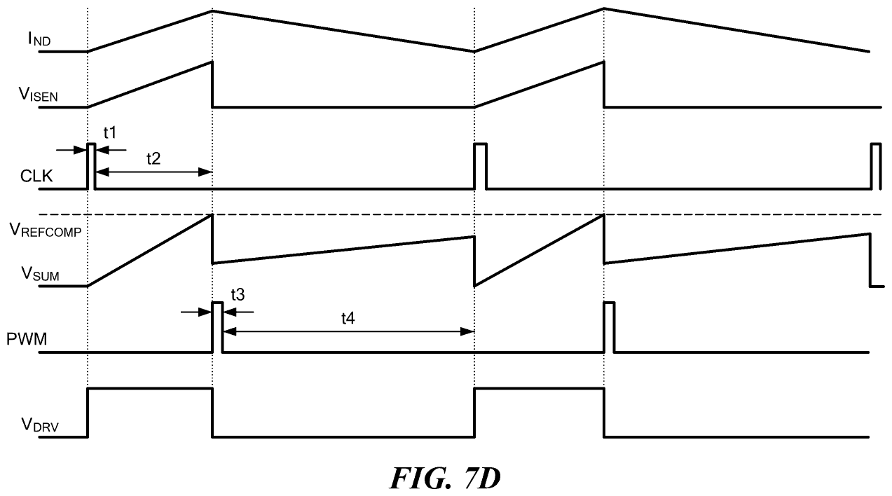

In the example of FIG. 3, the light source 110 is provided by a blue LED 210 having a wavelength around 450 nm. The LED 210 emits a pulsed intensity shown by graph 310 in a series of fixed intensity bursts or flashes. The light sensitive medium 120 is a luminescent sensing film of platinum porphyrin (Pt-porphyrin) or other luminescent material such as rubidium, and emits (or remits) a red light in a wavelength around 650 nm received by a red light photodetector 212. The luminescent sensing film, photoluminescent film, or simply thin film is a planar, sheet-like material having light emission properties as disclosed herein. The gaseous oxygen diffused 124′ during the blue light emission is shown in graph 324. It can be observed that an intensity 314 of the emitted red light varies inversely with the partial pressure (PO₂) 326 of diffused oxygen, responsive to constant intensity 312 pulses of blue light. The remitted red light varies based on a quenching effect of oxygen on an excitation of a luminophore in the light sensing medium 120, thus reducing the red light response as oxygen increases, as shown in dotted line region 314′ as the time t[high] transitions to t[low] in the presence of oxygen, described in further detail below. The luminescent material has the property of remitting light having an intensity and duration quenched by the presence of oxygen, therefore allowing oxygen measurement by observing the intensity and lifetime of the remitted light. The intensity/lifetime value of the remitted red light is received by a transimpedance amplifier 338 and converted by an analog/digital converter 339 for processing by logic 130. The intensity and lifetime are related as discussed below with respect to FIG. 9; in general, a lower oxygen presence mitigates the quenching effect and results in a greater intensity and lifetime of the remitted light. Measurement based on lifetime (duration) of the return pulses tends to be more resistant to factors such as LED fading/age and skin color variations.

FIG. 4 is a diagram of light emission energy received by the circuit of FIG. 3. Referring to FIGS. 2-4, in the excitation response of 314′, a quenching effect of oxygen on the photoluminescent sensing film 120′ is shown. The light sensitive medium 120 has a photoluminescent response and an oxygen based decay responsive the diffusion source for emitting a red light 121 inversely proportional with a partial pressure of oxygen in the gaseous diffusion 124. The sensing film 120′ includes an oxygen sensitive luminophore for exhibiting an emission of red light 121 in response to the blue light 111 based on a partial pressure of oxygen diffused through the light sensitive medium 120.

Benefits of the claimed approach will be apparent with reference to conventional approaches. Traditional devices measure PtcO₂ electrochemically, using methods requiring a heating element that increases the diffusion of O₂ from blood vessels, thus increasing the concentration of O₂ in the gas 124 above the targeted skin area. However, a heating element negatively affects the feasibility of a miniaturized PtcO₂ wearable as it substantially increases the wearable device size and the power requirement. In addition, the hotspot irritates and may even burn the skin during continuous monitoring.

To overcome such limitations, the disclosed approach employs a fluorescence-based method that allows the use of comfortable dry electrodes without the need for heating. This method uses the photoluminescent film including platinum porphyrin (Pt-porphyrin) or similar luminophore based medium. When a luminescent molecule absorbs a photon, it becomes excited from its ground state (S0) to some higher vibrational level of either the first or second electronic state (S1 or S2). When the film 120′ is exposed to blue light 411, it emits red light 421, the intensity and lifetime of which are inversely proportional to the concentration of O₂ 124 around the film as the energy level reaches S1 and following the excitation by the pulse of blue light, fall back to an energy level shown by S0. The fluorescence of the photoluminescent film is typically measured in terms of its lifetime (i.e. fall time) where to is the lifetime of the film fluorescence without the quencher (oxygen), and t0 is the lifetime of the fluorescence with the quencher. Conventional approaches refer to the so-called Stern-Volmer relationship in reference to the kinematics of quenching, discussed further below in FIG. 9.

The disclosed example includes a wearable or adhesive patch having the luminescent film through which patient-diffused oxygen passes. An oxygen presence passing through or adjacent the luminescent material causes the oxygen sensitive quenching response from the optical source. Various arrangements of luminescent materials in conjunction with the patient may be employed, along with corresponding photoreceptors and optical sources with light wavelengths (colors) based on the luminescent material. Similarly, targeted gases other than oxygen may be measured based on the luminescent film and gaseous sensitivity.

FIG. 5 is a graph of the light wavelengths emitted in the diagram of FIG. 4. Referring to FIGS. 3-5, the logic 130 is configured to compare the received red light 121 to a quenching effect of an oxygen concentration, such that the quenching effect increases with the oxygen concentration to indicate the partial pressure of oxygen in the diffused gases 124, and thus the oxygen levels in the blood and tissue underlying the patch 102. The light source 210 emits a blue light having an intensity 511, and the resulting excitation results in a red light remission having, for example an intensity of 521-1 when lower oxygen (PtcO₂), and increasing oxygen levels result in the red light intensity decreasing to levels of 521-2, 521-3, and to 521-4 as the increased oxygen quenches the red light response.

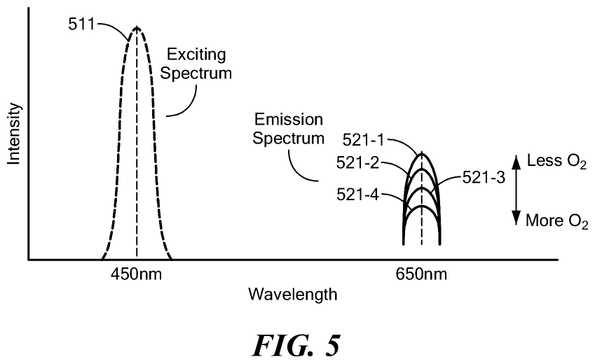

FIG. 6 is a block diagram of the wearable patch 102 as described in FIGS. 1-5, and FIGS. 7A-7D depict circuit schematics and graphs of the device 100. Referring to FIGS. 1-6 and 7A-7D, the circuit includes a power management portion 634 corresponding to the power supply 134, an LED driver 636 for powering and pulsing the blue LED 210, and an analog front end (AFE) 638 for receiving and generating a signal from the photodetector 212 corresponding to the remitted red light intensity indicative of the oxygen level. The logic 130 includes a mapping of luminescent diminution for an increasing oxygen availability, defined by a partial pressure of oxygen, that indicates the transcutaneous oxygen 124′ diffusing through the dermal (skin) surface 126. The quenching ability resulting from increasing oxygen 124′ causes the luminescent diminution, or intensity reduction, of the received red light 121 by the photodetector 212.

The power management portion 634 may be implemented as a Power Management Integrated Circuit (PMIC) including two bandgap references (BGR) 640, two power-on-reset (POR) blocks 642, two biasing circuits, and two low-dropout (LDO) regulators 644, powered off of an external 3 V battery. The BGR 640 includes 5 V CMOS devices to withstand a wide range of battery voltages (VBATT) and generates a stable reference voltage (VREF) of 1.2 V for the LDO, which converts the battery voltage to a stable 1.8 V supply voltage (VDD). A resistive feedback network sets the relationship between VREF and VDD. When the battery voltage drops below a certain level, the POR circuit provides a power-on reset signal for the LDO. The purpose of two LDO channels is to isolate the power path between the AFE and the LED driver and to distribute the load.

For example, the LED driver 136 excites the blue LED 210 with a peak wavelength of 450 nm, which excites the Pt-porphyrin film. The film emits red light of 650 nm, the intensity and lifetime of which are inversely proportional to the concentration of O₂. The current flowing through the photodetector 212 is proportional to the intensity of the red light from the film. Examples herein include Pt-porphyrin film having a sensitivity and responsiveness at the disclosed wavelengths. Alternate luminescent materials may of course be employed, such as rubidium based materials, and the wavelength values adjusted accordingly.

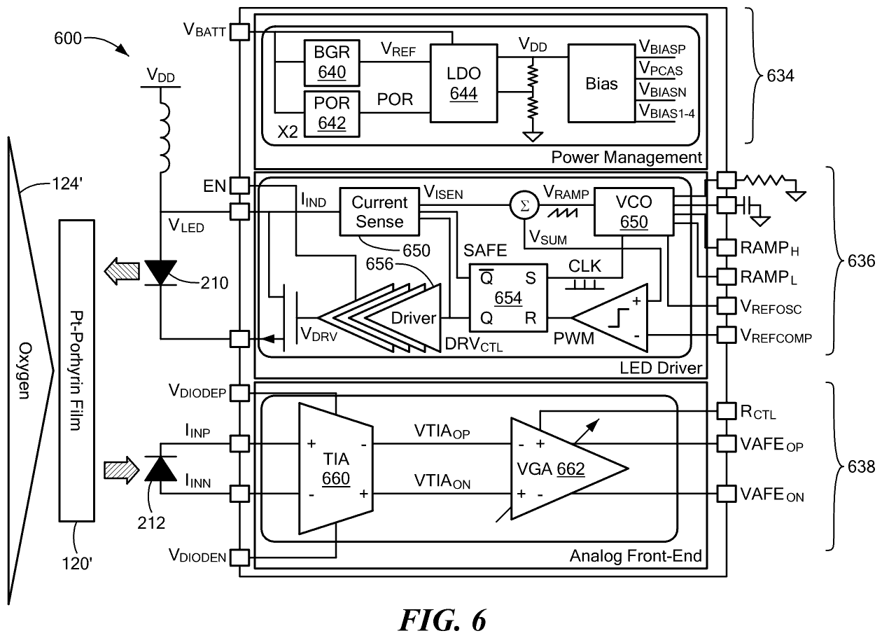

The LED driver 636 provides sufficient power to excite the blue LED with the proper intensity. It includes a current sensing block 650, a voltage-controlled oscillator (VCO) 652, a summer circuit, a comparator, an SR latch 654, and a driver 656, shown schematically in FIG. 7C. An off-chip inductor is employed to boost VDD to turn on the LED. The current sense block (FIG. 7A) measures the current (IIND) flowing through the inductor and outputs a signal (VISEN), which is then summed with the ramp signal (VRAMP) from the VCO, shown schematically in FIG. 7B, and compared with the current reference voltage VREFCOMP. This signal sets the limit of the maximum current in the inductor and keeps the driver stable. The timing diagram of the LED driver is illustrated in FIG. 7D.

The VCO generates the clock signal (CLK) and the ramp signal. VRAMP is summed with VISEN to create VSUM, which is then compared to an externally controlled reference voltage (VREFCOMP) in order to set the PWM signal. This signal resets the SR latch 654 and determines the pulse width of the DRVCTL signal. When EN is low, the driver is powered down, regardless of the DRVCTL. When enabled (EN=HIGH), VDRV controls the NMOS device based on the pulse-width modulated DRVCTL, and adjusts the current flowing through the LED. The EN signal is pulsed to reduce the power consumption of the readout. When EN is high, the LED driver pulls 16 mA current with VREFCOMP at 600 mV (increasing VREFCOMP increases IIND). When EN is low, the quiescent current of the LED driver 656, dominated by the current consumption of the VCO, is 180 mA. The externally controlled signals RAMPH, RAMPL, and VREFOSC, set the upper limit, the lower limit, and the frequency of the ramp waveform, respectively. Pulsation patterns of the blue light may vary, and are typically a series of rapid pulses interspersed between longer null intervals, as the oxygen diffusion has an inertial variance that can be effectively monitored periodically over 1-10 seconds to avoid excessive power drain.

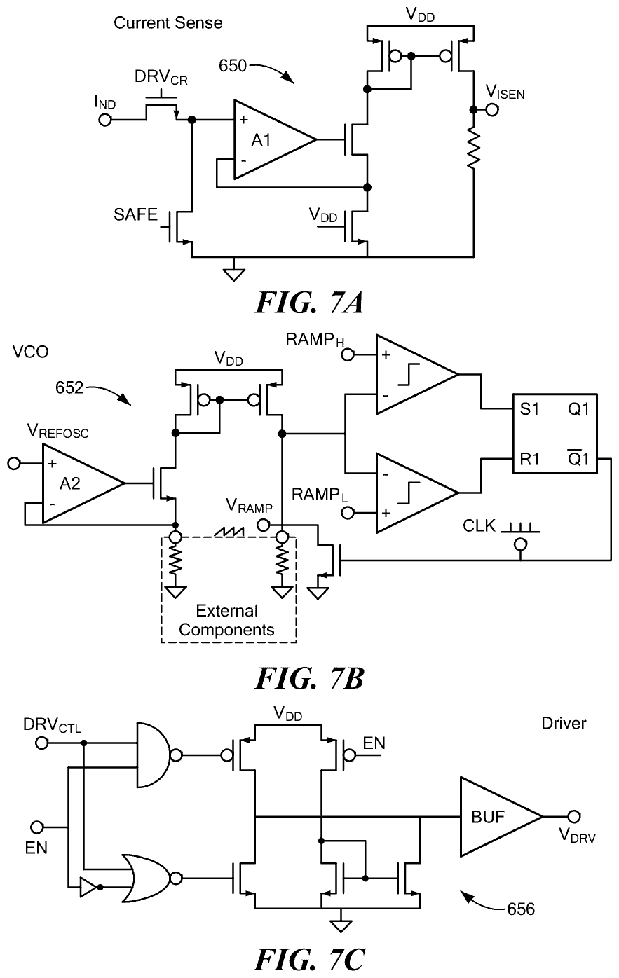

FIGS. 8A-8C shows a further detail of the analog front end 638 of FIG. 6. Referring to FIGS. 6-8C, in a particular arrangement, the AFE 638 includes two stages: a transimpedance amplifier (TIA) 660 and a variable gain amplifier 662 (VGA) 662. A differential TIA architecture minimizes the input impedance of the amplifier 662, which allows for small duty cycles and reduces common-mode noise. The cathode and the anode of the photodetector 212 connect to INP and INN inputs of the TIA 660, respectively.

The pulsing frequency of the system is based on the input impedance of the TIA and the capacitance of the photodetector 212. The TIA 660 may include current sensing 670-1, transimpedance 670-2, and common mode stages 670-3. The main purpose of the current-sensing stage 670-1 is to reduce the input impedance of the TIA with inner feedback loops created with amplifiers A7a and A7b and supplying a stable DC bias to the PD. The transimpedance stage 670-2 is designed to convert the current generated by the photodetector 212 to voltage with a gain of substantially around 50.1 k Ω using the feedback resistors R8 and R9 (shared with the common-mode feedback amplifier). The tunable gain is provided by the fully differential VGA 662, which follows the TIA. The variable gain is achieved by using pseudo resistors R2 and R3 controlled externally by RCTL.

FIG. 8C shows the schematic of the variable pseudo-resistors. The design consists of a two-stage NMOS op-amp with indirect feedback compensation A5, a single-stage differential amplifier A6, and non-tunable pseudo-resistors, R4, R5, R6 and R7. Varying the control voltage RCTL changes the resistance value of tunable pseudo-resistors from a few hundred kΩ to several hundred Go.

The intervals of light and the wavelengths thereof are depicted above in an example arrangement. Other intensities, pulsing cycles, and wavelengths may be provided and/or varied to produce the described results, possibly with alternate granularity and precision. The logic 130 in the example circuits my be provided by any suitable logic circuit, integrated circuit and/or programmed set of instructions, executed by a processor and/or embodied in a hardware or software rendering in volatile or non-volatile memory.

FIG. 9 shows a relation between intensity and duration of remitted light. Referring to FIG. 9, and using oxygen as an example as shown herein, a graph 900 shows measured emitted light output for various O₂ concentrations 910 in the environment. As the partial pressure of oxygen increases (arrow 920), the output decreases generally faster, with variations on pressure. A resulting curve 930 approximates data points as the lifetime (duration) 932 of the emitted fluorescent lifetime decreases as partial pressure of oxygen (PO₂) 934 increases, as per a Stern-Volmer response. In other words, the quenching effect of oxygen reduces both the intensity (strength) and lifetime (duration) that the emitted red light is detected. The relation of intensity to duration allows measurement of either or both towards computing the PO₂, however measurement of lifetime tends to be more robust, as described above.

An alternate configuration depicts a miniaturized wireless bimodal oxygenation status monitoring wearable device that will continuously monitor both the transcutaneous oxygen and peripheral blood oxygen saturation and also overcome the limitations of the traditional transcutaneous oxygen monitors such as requiring a heating element and a large, expensive bedside monitor. The portability and compact, nonintrusive form factor provide a viable personal device for remote monitoring of patient oxygen, which is particularly beneficial for patients with compromised respiratory systems. Healthcare providers may engage a remote network monitoring approach including a secure cloud data service that accompanies the wearable monitoring system. The patients will be provided with a monitor which will continuously record their partial pressure of oxygen (PtcO₂) and peripheral blood oxygen saturation (SpO₂). PtcO₂ readings not only incorporate new information and additional marker to assess the respiratory efficiency and oxygenation of blood and tissues, but may also be used to correct SpO₂ readings which are reported to be susceptible to skin-tone variations and motion artifacts. The PtcO₂ and SpO₂ readings will be sent to a server, where doctors can track the status of patients remotely.

Operationally, the overall system provides an affordable, wearable wireless sensor patch. The sensor patch can communicate via a wireless protocol to a base station which can either be medically monitored in a hospital setting or a smart device in a living environment. The data can be accessed in real-time. In addition to real time analysis, the data can also be securely stored. Post-processing of the data and customized machine learning algorithms can be applied to the stored data. This would help medical professionals conduct detailed analysis for better informed medical decisions and tailored treatment plans for the patient. This system can lead to critical observations about response to therapies and natural resolutions of different conditions. It is also an accessible solution that can help provide better care options to patients in remote locations.

The disclosed approach preferably implements a low-height profile, small form factor, low power consumption, and wireless communication capability for extended, long-term wear and comfort. The system must have comparable accuracy to contemporary “bedside” monitors. A significant feature of this sensing system is that it is a wearable wireless optical-based dual oxygenation monitoring system with no traditional heating element for transcutaneous oxygen measurement. This affords the patient improved freedom of movement, better skin compliance, and allows medical providers to receive more pertinent medical data to make better informed medical decisions and tailored treatment plans for the patients.

FIG. 10 shows a context diagram of the alternate configuration for combined partial pressure of both oxygen (PtcO₂) and peripheral blood oxygen saturation (SpO₂). In this alternate configuration, a sensor patch device 200 for blood gas measurement includes a substrate adapted for affixation to a patient epidermal surface, and a gaseous sensing film. A circuit provides for luminescent measurement of the gaseous sensing film for computing a gaseous concentration; and a wireless transmitter is adapted for sending a concentration measurement. The gaseous sensing film may be adapted for oxygen sensing, where the circuit is configured for measuring a partial pressure of transcutaneous oxygen (PtcO₂) and peripheral blood oxygen saturation (SpO₂). The substrate is engaged with a patient skin surface through any suitable means such as adhesion, plastic strap, or gravity.

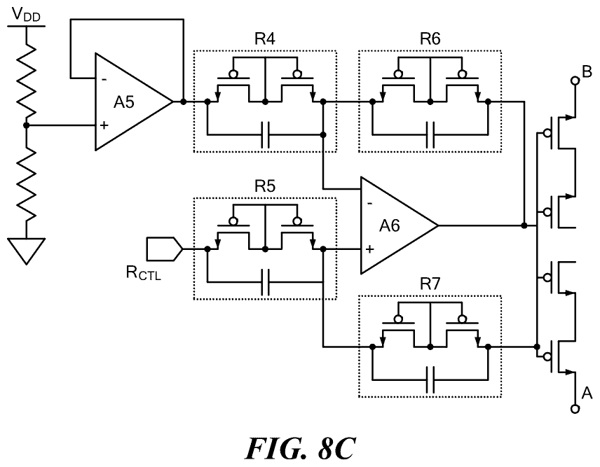

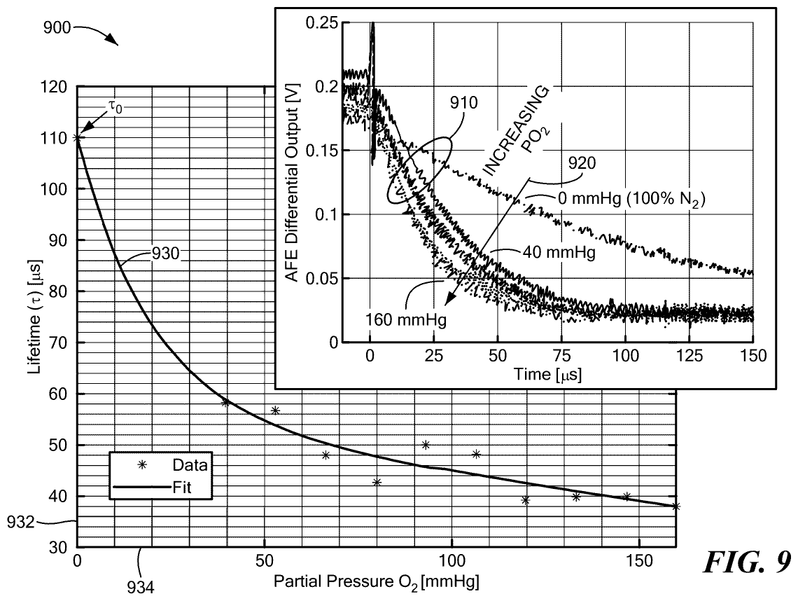

FIG. 10 demonstrates the fluorescence lifetime measured with the prototype monitor for different partial pressures of oxygen (PO₂) levels ranging from 0 mmHg to 418 mmHg. In FIG. 10, the lifetime of the luminescent emission as measured by the output of the PtcO₂ monitor for different values of partial pressure of oxygen and two distinct LED currents is shown. The results illustrate that the fluorescence lifetime is inversely proportional to the partial pressure of oxygen. FIG. 11 shows a difference between measured lifetimes. As shown in FIG. 11, there is a continuous inaccuracy of less than 0.25 μs between recorded lifetime readings of the same PO₂ values, except in the 0 mm Hg case, where the lifetime value is the highest. This result shows that the lifetime-based technique used in this prototype is resistant to changes in the optical path. It is also important to note that the monitor exhibits good sensitivity in the clinically relevant range for healthy humans, 50-150 mmHg. FIG. 12 shows the transient response of the PtcO₂ monitor to the step changes in the partial pressure of oxygen (the vertical dashed lines indicate the times when the value of partial pressure of oxygen increased by 38 mmHg, as shown in the top of the graph). FIG. 12 therefore demonstrates the transient lifetime response of the prototype to step changes in PO₂. The prototype was responsive to the changes in the lifetime value, corresponding to the step increases in PO₂. These successful measurements are proof of the feasibility of detecting changes in the O₂ concentration.

Following the above gas testbench experiments, configurations also demonstrate the ability of a prototype to respond to biological changes in the human body through human subject tests. For this purpose, tests were performed at the fingertips and forearms of three healthy volunteers (two males and one female). We followed two distinct methods, namely pressure-induced arterial occlusion and hypoxic gas delivery, to modulate the partial pressure of O₂ diffusing through the skin to examine the prototype's response to biological changes in the body. To decrease the blood circulation through occlusion, the volunteers either clenched their index finger with the fingers of the opposite hand or a pressure cuff can be placed on their upper arm. In the latter, we inflated the cuff pressure to 180 mmHg to induce arterial occlusion, resulting in a decrease in the volunteer's PtcO₂. To induce hypoxia, an altitude generator with a mask on the volunteer's face was employed. The altitude generator reduced the PO₂ in the air in its reservoir and inhaled by the volunteer. The reduced O₂ intake caused an overall decrease in the arterial partial pressure of oxygen (PaO₂) in the body, leading to a decrease in the volunteer's PtcO₂.

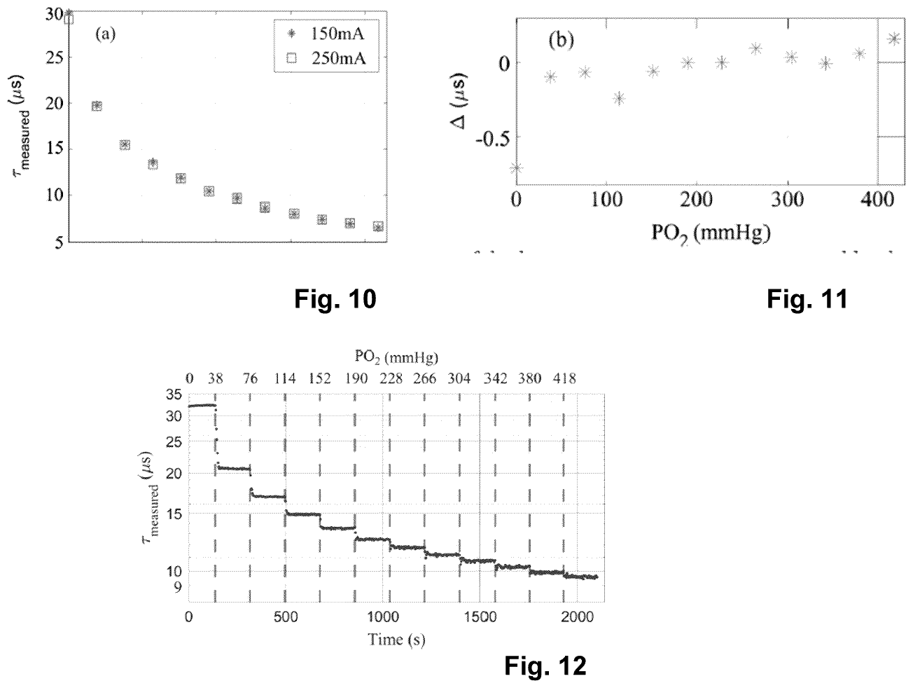

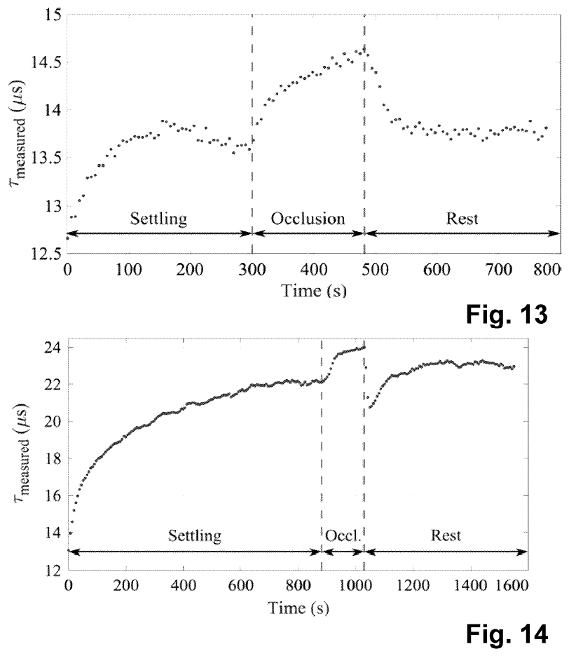

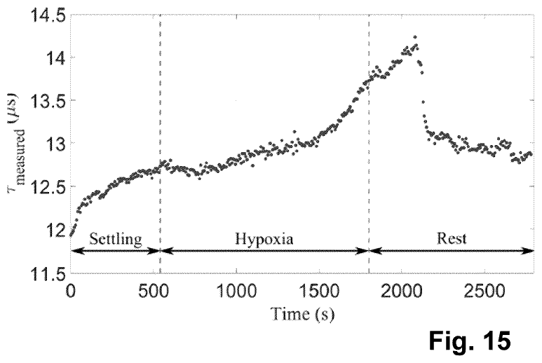

We conducted the first test on the first volunteer's finger by applying occlusion through clenching the index finger for three minutes. The results of this test are demonstrated in FIG. 13. The lifetime stabilized around 13.6 μs in five minutes. Following stabilization, arterial occlusion was applied for three minutes, and the lifetime increased to 14.6 μs as PtcO₂ dropped. After releasing the pressure on the finger, the lifetime immediately began to decrease in the rest phase, lasting five minutes, as oxygenated blood began to flow. Eventually, the lifetime stabilized at around 13.8 μs. We conducted the second test on the forearm of the second volunteer by applying occlusion. Arterial occlusion was induced using a pressure cuff. The results of this test are demonstrated in FIG. 14. The lifetime stabilized at around 22.1 μs in 15 minutes. An increase in the lifetime to 24 μs was observed after preventing the influx of oxygenated blood for two minutes in the occlusion phase. After releasing the pressure on the cuff, the lifetime dropped sharply before returning to slightly above the base value. This rapid drop in lifetime may be explained by the fact that when the pressure on the forearm is released, there is a rush of oxygenated blood into the forearm, increasing PaO2 above the value before occlusion. The lifetime stabilized at around 23 μs after blood flow was restored, slightly higher than the base value. Finally, we conducted the hypoxia test on the third volunteer from the fingertip, shown by the results of FIG. 15. The lifetime stabilized at around 12.7 μs in nine minutes. During the hypoxic gas delivery, the lifetime gradually increased to 13.6 μs in 21 minutes. It should be noted that the hypoxic gas delivery changed the O2 amount in the whole body, leading to a slow and delayed metabolic response. After removing the mask in the rest phase, an increase in the lifetime to 14.1 μs was observed due to body's delayed response to the changes. Then, the lifetime started to decrease and stabilized around 12.8 μs, over the course of 16 minutes. The above figures and description demonstrate the viability and association of the bimodal SpO₂ and PtcO₂ over the measurement of only PtcO₂ discussed with respect to FIGS. 1-9.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. An epidermal, portable wireless transcutaneous sensing device, comprising:
a saturation sensor configured to detect a peripheral blood oxygen saturation (SpO₂) based on an optical response from the skin surface;
a transcutaneous sensor configured to detect a partial pressure of transcutaneous oxygen (PtcO₂) based on an optical response from a skin surface; and
a transmitter for communication with a remote server configured for rendering values corresponding to the transcutaneous oxygen and peripheral blood oxygen saturation.

2. The device of claim 1 wherein the saturation sensor further comprises:
a plurality of illumination sources;
a plurality of photodetectors; and
sensing logic configured to:
activate the illumination sources to irradiate the skin surface;
monitor the photodetectors to receive reflected illumination; and
compute the peripheral blood oxygen saturation based on the reflected illumination.

3. The device of claim 1 further comprising
a skin mounted form factor, further comprising:
a substrate configured for epidermal mounting;
a first circuit including the saturation sensor;
a second circuit including the transcutaneous sensor;
a power supply connected to the first and second circuits and the transmitter; and
a system-on-chip (SoC) containing the first circuit, the second circuit and the transmitter.

4. The device of claim 2 wherein the saturation sensor further comprises:
a red LED (light emitting diode);
an infrared (IR) LED;
two photodetectors, the photodetectors configured for measuring respective resulting photoplethysmography (PPG) signals.

5. The device of claim 4 wherein the sensing logic is configured to excite the red LED and the IR LED for inducing reflections based on metabolic variations in the skin surface, the two photodetectors responsive to the reflections.

6. The device of claim 1 further comprising an interface to a public access network, the remote server accessible via the public access network and further configured for storing and coalescing a series of respective values indicative of the SpO₂ and the PtcO₂ for generating an indication of respiratory health.

7. The device of claim 1 wherein the transcutaneous sensor further comprises:
an emitter adapted to project light of a predetermined sensory wavelength;
a photoluminescent film opposed to the emitter for receiving the light from the emitter and responsive to emit light of a sensed wavelength in response to the received light based on a gaseous exposure of the photoluminescent film;
an optical sensor responsive to the sensed wavelength for returning a signal indicative of the sensed wavelength; and
a correlation circuit for computing a gaseous presence based on the signal.

8. The device of claim 7 wherein the emitter and the optical sensor are disposed in a common plane and opposed to a plane defining the photoluminescent film.

9. The device of claim 7 further comprising a system-on-chip (SoC) semiconductor structure including a sensing circuit, the sensing circuit orienting the emitter, optical sensor and correlation circuit in adapted for engagement with an epidermal surface.

10. The device of claim 7 wherein the photoluminescent film, emitter and the optical sensor define a layered arrangement with an epidermal surface, the layered arrangement disposing the photoluminescent film opposed from the emitter and optical sensor, and between an epidermal surface and the emitter and optical sensor.

11. The device of claim 7 wherein the photoluminescent film is disposed in planar communication with an epidermal surface and responsive to transcutaneous gases passing from the epidermal surface to the photoluminescent film.

12. The device of claim 7 wherein the signal is indicative of a luminescence intensity of the sensed wavelength, and the correlation circuit includes concentration logic for computing a concentration of a predetermined substance defined by the gaseous presence.

13. A system for bimodal sensing of a patient oxygen level using a system-on-chip (SoC) substrate, comprising:
a saturation sensor configured to detect a peripheral blood oxygen saturation (SpO₂) based on an optical response from the skin surface;
a transcutaneous sensor configured to detect a partial pressure of transcutaneous oxygen (PtcO₂) based on an optical response from a skin surface; and
a transmitter for communication with a remote server configured for rendering values corresponding to the transcutaneous oxygen and peripheral blood oxygen saturation.

14. The system of claim 13 further comprising:
an interface to a public access network, the remote server accessible via the public access network and further configured for storing, in a memory, the wherein the transmitter is configured for storing and coalescing a series of respective values indicative of the SpO₂ and the PtcO₂ for generating an indication of respiratory health.
